(19) United States
(12) Patent Application Publication
(10) Pub. No.: US 2006/0013803 A1
(43) Pub. Date: Jan. 19, 2006

(54) Modified dendritic cells

(76) Inventor: Lung-Ji Chang, Gainesville, FL (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(21) Appl. No.: 11/122,456

(22) Filed: May 5, 2005

Related U.S. Application Data
(63) Continuation-in-part of application No. PCT/US03/35482, filed on Nov. 7, 2003.
(60) Provisional application No. 60/424,602, filed on Nov. 7, 2002.

(51) Int. Cl. A61K 48/00; C12N 15/867

(52) U.S. Cl. 424/93.2; 435/456

(57) ABSTRACT

Infection of a dendritic cell with a lentivirus impairs the dendritic cell's ability to act as an antigen presenting cell that polarizes a naïve T cell to develop along the Th1 pathway. This impairment is restored by infecting dendritic cells with lentiviruses containing vectors encoding IL-7, IL-12, and siRNA targeting IL-10 RNA.

### CROSS-REFERENCE TO RELATED APPLICATIONS

The present application claims the priority of U.S. provisional patent application Ser. No. 60/424,602 filed Nov. 7, 2002 and entitled “Modulation of Dendritic Cell Function.”

### STATEMENT AS TO FEDERALLY SPONSORED RESEARCH

The invention was made with U.S. government support under grant number P50 HL59412 awarded by the National Institutes of Health. The U.S. government may have certain rights in the invention.

### FIELD OF THE INVENTION

The invention relates to the fields of molecular biology, gene therapy, immunology, and virology. More particularly, the invention relates to compositions and methods for transducing dendritic cells with lentiviral vectors (LVs) to modulate dendritic cell function.

### BACKGROUND OF THE INVENTION

Although LVs such as human inmunodeficiency virus (HIV) are associated with disease in animals, their ability to transfer exogenous nucleic acid into a host cell has been exploited in gene therapy experiments designed to treat diseases. For gene therapy applications, LVs offer several advantages over other vectors. For example, LVs derived from HIV employ cell entry and genome integration processes similar to those of the wild-type virus, including the ability to infect both dividing and non-dividing cells. The advantage of infecting both dividing and non-dividing cells makes LVs a very popular gene transfer vehicle compared with the conventional oncoretroviral vectors. The efficient integration, the broad host cell tropism and low tissue specificity make LVs more efficient and useful than other vectors such as adeno-associated virus vectors.

LVs have been used to transfer genes into dendritic cells (DC) for use in immunotherapy and vaccine applications. DC, professional antigen presenting cells, have been popular in such applications because of their ability to induce a vigorous T cell response. As reported below, however, it was discovered that DC transduced with LVs displayed a diminished ability to activate naive T cells. After LVs transduction, DC showed altered cytokine response and surface marker expression, including up-regulation of IL-10 and down-regulation of T cell costimulatory molecules. In line with these findings, DC transduced with LVs were compromised in their ability to polarize naive T cells to Th1 effectors—an effect that may limit the use of LVs-transduced DC in immunotherapy and vaccine applications.

### SUMMARY

The invention relates to the discovery of methods and compositions for overcoming LVs-induced impairment of DC function. In making the invention, a series of immune modulatory strategies were investigated to overcome the DC-induced T cell dysfunction caused by HIV/lentiviral infection, including applications of soluble cytokines and immune modulators. By delivering immune modulators such as lentiviral immunomodulatory viruses to DCs, the DC and T cell dysfunctions caused by HIV (lentiviral) infection can be corrected. Specifically, the impaired Th1 response is restored by infecting DC with lentiviruses containing vectors encoding IL-7, IL-12, or siRNA targeting IL-10 RNA. This technology provides specific immunotherapeutic formulas for overcoming the immune-suppression problems associated with HIV infection of DC during treatment, vaccination or vector applications in patients.

Accordingly, the invention features a nucleic acid including a first nucleotide sequence derived from a lentivirus and a second nucleotide sequence that encodes IL-7, IL-12, or an siRNA specific for IL-10. Also within the invention is a dendritic cell (e.g., one infected with a lentivirus) into which has been introduced a purified nucleic acid comprising a nucleotide sequence that encodes an agent selected from the group consisting of IL-7, IL-12, and an siRNA specific for IL-10.

Another aspect the invention features a method of modulating the T cell activating ability of a dendritic cell. The method includes the step of modulating the amount of IL-7, IL-10, and/or IL-12 associated with the dendritic cell. For example, this step can involve increasing the amount of IL-7 and/or IL-12 associated with the cell, and/or decreasing the amount of IL-10 associated with the cell. Modulating the amount of a cytokine associated with a dendritic cell can be achieved by contacting the cell with a soluble cytokine, removing a soluble cytokine from the cell, or by introducing into the cell a purified nucleic acid encoding the cytokine or an agent that reduces expression of the cytokine (e.g., an siRNA or an anti-sense nucleic acid).

As used herein, phrase “nucleic acid” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules.

As used herein, the term “vector” refers to an entity capable of transporting a nucleic acid and/or a virus particle, e.g., a plasmid or a viral vector.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of virology terms can be found in Granoff and Webster, Encyclopedia of Virology, 2nd edition, Academic Press: San Diego, Calif., 1999; and Tidona and Darai, The Springer Index of Viruses, 1st edition, Springer-Verlag: New York, 2002. Commonly understood definitions of microbiology can be found in Singleton and Sainsbury, Dictionary of Microbiology and Molecular Biology, 3rd edition, John Wiley & Sons: New York, 2002.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### BRIEF DESCRIPTION OF THE DRAWINGS

FIG. 1 is a highly schematic diagram showing various vector constructs used in the invention.

FIG. 2 is a highly schematic diagram showing siRNAs specific for IL-10 RNA.

### DETAILED DESCRIPTION

The invention provides methods and compositions for overcoming an LV-induced impairment of a DC's T cell activating ability. The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Nucleic Acids/LVs

The invention provides a nucleic acid that includes a first nucleotide sequence derived from a lentivirus and a second nucleotide sequence that encodes an agent capable of modulating DC function (e.g., overcoming a LV-induced T cell activation impairment). The nucleic acids of the invention preferably take the form of a LV. A number of different types of LVs are known including those based on naturally occurring lentiviruses such as HIV-1, HIV-2, simian immunodeficiency virus (SIV), feline immunodeficiency virus (FIV), bovine immunodeficiency virus (BIV) and others. See U.S. Pat. No. 6,207,455. Although the invention is described using HIV-1 based vectors, other vectors derived from other lentiviruses might also be used by adapting the information described herein. Because of the many advantages HIV-1 based vectors provide for gene therapy applications, these are presently preferred.

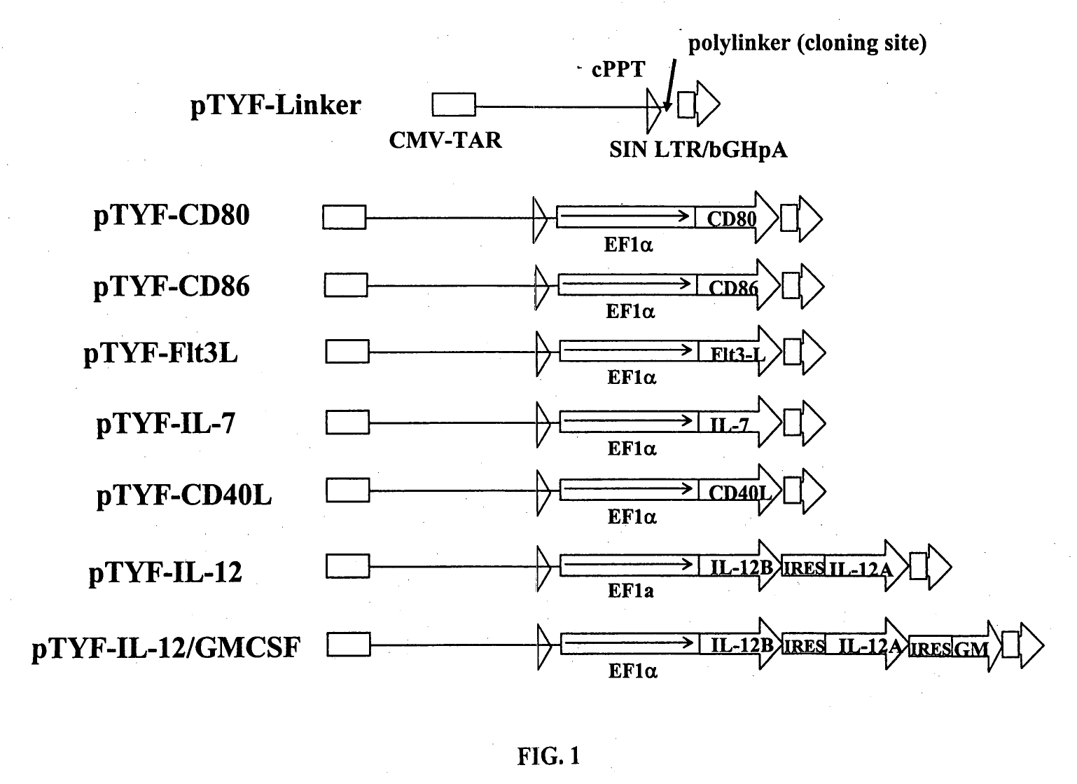

The LVs of the invention might be pseudotyped, e.g., to overcome restricted host cell tropism. For example, LVs pseudotyped with vesicular stomatitis virus G (VSV-G) viral envelopes might be used. To enhance safety, a self-inactivating (SIN) LV might also be used. For example, a SIN LVs can be made by inactivating the 3′ U3 promoter and deleting of all the 3′ U3 sequence except the 5′ integration attachment site which is important for the integration into host chromosome. A particularly preferred construct for designing vectors of the invention is pTYF shown in FIG. 1.

The second nucleotide sequence that encodes an agent capable of modulating DC function can be one encoding a cytokine such as IL-7 or IL-12 (both shown herein to overcome LVs-induced DC impairment). Lentiviruses containing LVs encoding IL-12, IL-12+GM-CSF, and IL-7 are used to modulate DC function (e.g., correct the impaired Th1 response by lentivirus-infected DC). Preferred LVs include pTYF-IL-12 bi-cistronic vectors, pTYF-IL12-GM-CSF tri-cistronic vectors, and pTYF-IL-7. Preferred lentiviruses of the invention contain LVs pTYF-IL-12 bi-cistronic vectors, pTYF-IL12-GM-CSF tri-cistronic vectors, and pTYF-IL-7 and are pseudotyped with VSV-G to broaden their host cell tropism (see Chang and Gay, Current Gene Therapy 1, 237-251, 2001; Chang and He, Curr Opin Mol Ther 3(5), 468-75, 2001).

The viral vectors (and corresponding viruses) used in the experiments described herein are MLV-based and SIN lentiviral (HIV-1)-based vectors. FIG. 1 shows the structures of the LVs pTYF-CD80, pTYF-CD86, pTYF-Flt3-L, pTYF-IL-7, pTYF-CD40L, pTYF-IL-12, and pTYF-IL-12/GMCSF. The starting plasmid for cloning the SIN LVs is pTYF, a SIN vector featuring a central polypurine tract (cPPT). Inclusion of a cPPT sequence has been shown to enhance viral vector activity approximately 3-fold. The SIN LVs also contain a 3′ bovine growth hormone polyadenylation signal (bGHpA) inserted behind a 3′ truncated long terminal repeat (LTR). The SIN LVs encode a number of cytokines, including IL-12, IL-12 plus GM-CSF and IL-7, as well as immune modulatory molecules such as CD80 or CD86 (Liang and Sha, Curr. Opin. Immunol. 14:384-390, 2002; and Carreno and Collins, Annu. Rev. Immunol 20:29-53, 2002), and Flt3-L. Human cytokine cDNA sequences contained within viral vectors are amplified by RT-PCR from human peripheral blood lymphocytes (CD80, CD86, GM-CSF, IL-12 and IL-7), or from human tumor cells (TE671 cells for Flt3-ligand). The IL-12 gene has two components, IL-12A and IL-12B. For use in modulating DC function, cDNAs of both IL-12 components are cloned simultaneously into a bi-cistronic vector with an internal ribosome entry site (IRES) between these two cDNAs. For the pTYF-IL-12-GMCSF vector, two different IRES elements are placed between IL-12B/IL-12A, and IL-12A/GM-CSF cDNAs to generate a tri-cistronic expression vector. Genes within the viral vectors can be under the control of any suitable promoter (e.g., a strong promoter such as human elongation factor 1 alpha, EF1a). For construction of pTYF vectors, see Zaiss et al., J. Virology 76:7209-7219; and Chang et al., Gene Therapy 6:715-728. The MLV vectors (and corresponding viruses) were constructed as described in Zaiss et al., J. Virol. 76:7209-7219, 2002.

Construction of recombinant LVs and virions is discussed in Buchschacher et al., Blood 95:2499-2504, 2000; Chang et al., Gene Therapy 6:715-728, 1999; Emery et al., PNAS 97:9150-9155, 2000; Naldini et al., Science 272:263-267, 1996; Paillard et al., 9:767-768, 1998; Sharma et al., PNAS 93:11842-11847, 1996; Reiser et al., PNAS 93:15266-15271, 1996; and Chinnasamy et al., Blood 96:1309-1316, 2000. SIN vector design is described in Miyoshi et al., J. Virol. 72:8150-8157, 1998; Zufferey et al., J. Virol. 72: 9873-9880, 1998; Iwakuma et al., Virology 261:120-132, 1999; Mangeot et al., J. Virol. 74:8307-8315, 2000; and Schnell et al., Hum. Gene Ther. 11:439-447, 2000.

### Dendritic Cells

The invention provides a DC into which has been introduced a purified nucleic acid having a nucleotide sequence that encodes an immunomodulatory agent such as IL-7, IL-12, or an siRNA specific for IL-10. DCs that might be used include mammalian DCs such as those from mice, rats, guinea pigs, non-human primates (e.g., chimpanzees and other apes and monkey species), cattle, sheep, pigs, goats, horses, dogs, cats, and humans. The DCs may be those within a mammalian subject (i.e., in vivo), or those within an in vitro culture (e.g., those cultured in vitro for ex vivo delivery to a subject). DCs according to the invention contain a nucleic acid a purified nucleic acid having a nucleotide sequence that encodes an immunomodulatory agent such as IL-7, IL-12, or an siRNA specific for IL-10. In preferred DCs, the nucleic acid is expressed, resulting in a polypeptide or RNA.

DCs can be obtained from any suitable source, including the skin, spleen, bone marrow, or other lymphoid organs, lymph nodes, or blood. Preferably, DCs are obtained from blood or bone marrow for use in the invention. Typically, DCs are generated from bone marrow and peripheral blood mononuclear cells (PBMC) after stimulation with exogenous granulocyte-macrophage colony stimulating factor (GM-CSF) and interleukin-4. Methods for obtaining DCs from bone marrow cells and culturing DCs are described in Inaba et al., J. Exp. Med. 176:1693-1702, 1992; and Bai et al., Int. J. Oncol. 20:247-253, 2002. Methods for culturing DCs from hematopoietic progenitor cells (Mollah et al., J. Invest. Dermatol. 120:256-265, 2003) and monocytes (Nouri-Shirazi and Guinet Transplantation 74:1035-1044, 2002) are also known in the art. An example of a large-scale monocyte-enrichment procedure for generating DCs is described in Pullarkat et al. (J. Immunol. Methods 267:173-183, 2002). DCs may be isolated from a heterogeneous cell sample using DC-specific markers in a fluorescence-activated cell sorting (FACS) analysis (Thomas and Lipsky J. Immunol. 153:4016-4028, 1994; Canque et al., Blood 88:4215-4228, 1996; Wang et al., Blood 95:2337-2345, 2000). Immature DC are characterized by low level expression of costimulatory molecules, CD80/86, CD40; poor ability to induce T cell activation; inability to produce IL-12p70; and the potential to induce regulatory or anergic T cells. In comparison, mature DC produce IL-12p70 and express high levels of MHC class II antigens, CD80/86, and CD40, IL-12p70 production. A population of cells containing DCs as well as isolated DCs may be cultured using any suitable in vitro culturing method that allows growth and proliferation of the DCs.

### Modulating DC Function

The invention also provides methods for modulating DC function. DCs stimulate naive T helper cells to differentiate into either IFN-gamma-producing Th1 or IL-4-producing Th2 effector cells, which mediate different immune responses. Distorted Th responses result from transduction of DC with LVs and by infection with lentiviruses. In particular, lentiviral-transduced and lentivirus-infected DC induce differentiation of naive Th cells toward an impaired Th1 response and an enhanced IL-4-producing Th2 response. Compositions and methods of the invention can be used to improve the immune-activating capacity of DCs (e.g., restoring the Th1 response) by providing cytokines (e.g., immunogenes) to DCs. Examples of suitable cytokines include IL-12 and IL-7. Other cytokines that enhance a Th1 response may also be used in the invention.

To modulate DC function (e.g., restore a Th1 response), a DC cell is contacted with a LV that contains a purified nucleic acid including a nucleotide sequence derived from a lentivirus and at least one transgene not derived from a lentivirus. The transgene may be any cytokine that enhances a Th1 response, including IL-12 and IL-7.

In one example of modulating DC function, DC are infected with lentiviruses containing vectors encoding IL-12, IL-12 plus GM-CSF and IL-7. In this example, immature DC are infected with Mock (293T supernatants), TYF-PLAP, TYF-IL-12, TYF-IL12-GM-CSF, or TYF-IL-7. After maturation with LPS (80 ng/ml) plus TNF-alpha (20 u/ml) for 24 hr, the DCs are harvested and co-cultured with naive CD4+ T cells at a DC/T ratio of 1:20. After 5 days of co-culture, the T cells are expanded in the presence of IL-2 (25 u/ml) for an additional 7 days. Th1, Th2 and Th0 populations are then measured by intracellular IFN-gamma and IL-4 staining after 6 hr of restimulation with ionomycin and PMA in the presence of Brefeldin A. LVs encoding immune modulatory molecules such as IL-12, IL-12+GM-CSF, and IL-7 can effectively correct the impaired Th1 response by lentivirus infected DC.

### Modulating an Immune Response in a Subject

Compositions and methods for increasing and decreasing an immune response in a subject may be used in a variety of DC-based immunotherapy strategies for treating a many different disorders. Mature DC are the key antigen presenting cell population which efficiently mediates antigen transport to organized lymphoid tissues for the initiation of T cell responses (e.g., induction of cytotoxic T lymphoctyes). The normal function of DCs is to present antigens to T cells, which then specifically recognize and ultimately eliminate the antigen source. DCs are used as both therapeutic and prophylactic vaccines for cancers and infectious diseases. Such vaccines are designed to elicit a strong cellular immune response. DC biology, gene transfer into DC, and DC immunotherapy are reviewed in Lundqvist and Pisa, Med. Oncol. 19:197-211, 2002; Herrera and Perez-Oteyza, Rev. Clin. Esp. 202:552-554, 2002; and Onaitis et al., Surg. Oncol. Clin. N. Am. 11:645-660, 2002.

The induction of cytotoxic and type 1 helper (Th1) cellular responses is highly desirable for vaccines targeting chronic infectious diseases or cancers (P. Moingeon, J. Biotechnol. 98:189-198, 2002). The use of modified DCs expressing interleukins that upregulate Th1 cells and their actions may be used to increase resistance to pathogens (J. W. Hadden, Int. J. Immunopharmacol. 16:703-710, 1994). For the treatment of HIV infection, for example, DCs can be targeted both ex vivo and in vivo to initiate and enhance HIV-specific immunity (Piguet and Blauvelt J. Invest. Dermatol. 119:365-369, 2002).

In addition to HIV therapies, modified DCs of the invention may be used in cancer immunotherapies. DCs manipulated to present tumor antigen to secondary lymphoid organs and resting, naive T-cells are useful for generating tumor-specific T-cells (A. F. Ochsenbein Cancer Gene Ther. 9:1043-1055, 2002). For example, DCs modified to express a myeloma-associated antigen may be useful as an anticancer therapy for multiple myeloma (Buchler and Hajek Med. Oncol. 19:213-218, 2002). DCs expressing certain cytokines or chemokines have been shown to display a substantially improved maturation status, capacity to migrate to secondary lymphoid organs in vivo, and ability to stimulate tumor-specific T-cell responses and induce tumor immunity in vivo. DCs modified to express cytokines, therefore, may be useful for inducing tumor immunity and may be used in combination with DC modified to express tumor antigens. The therapeutic role of DCs in cancer immunotherapy is reviewed in Lemoli et al., Haematologica 87:62-66, 2002; A. F. Ochsenbein, Cancer Gene Ther. 9:1043-1055, 2002; Zhang et al., Biother. Radiopharm. 17:601-619, 2002; Di Nicola et al., Cytokines Cell Mol. Ther. 4:265-273, 1998; D. Avigan, Blood Rev. 13:51-64, 1999, and Syme et al., J. Hematother. Stem Cell Res. 10:601-608, 2001.

In an example of a DC-based vaccine strategy, LV encoding an immunogen are used to modify DCs, resulting in expression and presentation of the immunogen to resting, naive T-cells. Such an antigen presentation strategy can be used alone or in association, as part of mixed immunization regimens, in order to elicit broad immune responses. Different strategies of immunization involving delivery of DCs to patients are described in Onaitis et al., Surg. Oncol. Clin. N. Am. 11:645-660, 2002.

Modified DCs may also be used to modulate T-cell (Th1 and/or Th2) responses for the treatment of autoimmune disorders (e.g., arthritis, asthma, atopic dermatitis). The balance between Th1 and Th2 cells is of importance in many autoimmune disorders. Th1 cell activity predominates in joints of patients with rheumatoid arthritis and insulin-dependent diabetes mellitus, whereas Th2 cell-dominated responses are involved in the pathogenesis of atopic disorders (e.g., allergies), organ-specific autoimmune disorders (type 1 diabetes and thyroid disease), Crohn's disease, allograft rejection (e.g., acute kidney allograft rejection), and some unexplained recurrent abortions (Allergy Asthma Immunol. 85:9-18, 2000). Allograft rejection occurs when the host immune system detects same-species, non-self antigens. To prevent or treat allograft rejection, modified DCs may be used to induce tolerance to tissue-specific antigens (B. Arnold Transpl. Immunol. 10:109-114, 2002). DC expressing immunosuppressive molecules may also be used as a therapy for allograft rejection (Lu and Thomson Transplantation 73:S19-22, 2002).

Modified DCs may further be used to induce an immune response against a microbial pathogen (e.g., viruses, bacteria, fungi, protozoa, and helminths). For example, DCs might be modified to express a peptide antigens derived from the microbial pathogen. Presentation of the antigen by such DC could stimulate a vigorous immune response against the pathogen.

### EXAMPLES

The present invention is further illustrated by the following specific examples. The examples are provided for illustration only and are not to be construed as limiting the scope or content of the invention in any way.

### Example 1

### Materials and Methods

Generation of monocyte-derived dendritic cells. Peripheral blood mononuclear cells (PBMC) were isolated from buffy coats of healthy donors (Civitan Blood Center, Gainesville, Fla., USA) by gradient density centrifugation in Ficoll-Hypaque (Sigma-Aldrich, USA) as previously described (Chang and Zhang, Virology 211:157-169, 1995). DC were prepared from PBMC according to Thurner et al. (J. Immunol. Methods 223:1-15, 1999) with the following modifications. On day 0, five million PBMC per well were seeded into twelve-well culture plates in serum-free AIM-V medium. After incubation at 37° C. for 1 h, non-adherent cells were gently washed off and the remaining adherent monocytic cells were further cultured in AIM-V medium until day 1. The culture medium was removed carefully not to disturb the loosely adherent cells, and new AIM-V medium (1 ml per well) containing recombinant human GM-CSF (560 u/ml, Research Diagnostic Inc. Flanders N.J.) and IL-4 (25 ng/ml, R&D Systems) was added and the cells were cultured in a 37° C., 5% CO₂ incubator. On day 3, 1 ml fresh AIM-V medium containing GM-CSF (560 u/ml) and IL-4 (25 ng/ml) was added to the culture. On day 5, the non-adherent cells were harvested by gentle pipetting. After wash, the DC were frozen for later use or used immediately.

Lentiviral transduction of immature DC and DC maturation. The day 5 immature DC were plated at 5×10⁵ per well in a 24-well plate containing 200 ul of medium supplemented with GM-CSF (560 u/ml) and IL-4 (25 ng/ml). Transduction of DC was carried out by adding concentrated LVs to the cells at an multiplicity of infection (MOI) of 50-100. The cells were incubated at 37° C. for 2 hr with gently shaking every 30 min, and then 1 ml DC medium was added and the culture was incubated with the viral vectors for additional 12 h. DC maturation was induced by adding lipopolysaccharide (LPS) at final concentration 80 ng/ml and TNF-alpha at final concentration 20 u/ml to the DC culture for 24 h. The matured DC were harvested after incubation with AIM-V medium containing 2 mM EDTA in a 37° C., 5% CO2 incubator for 20 min. The cells were washed three times and used for subsequent experiments.

Antibody staining and flow cytometry. For analysis of cell surface marker expression by flow cytometry, the DC were incubated for 10 min with normal mouse serum and then 30 min with fluorochrome-conjugated anti-human monoclonal antibodies, including HLA-ABC (Tu149, mouse IgG2a, FITC-labeled, Caltag Laboratories), HLA-DR (TU36, mouse IgG2b, FITC-labeled, Caltag Laboratories), CD1a (HI49, mouse IgG1k, APC-labeled, Becton Dickinson), CD80 (L307.4, mouse IgG1k, Cychrome-labeled, Becton Dickinson), CD86 (RMMP-2, Rat IgG2a, FITC-labeled, Caltag Laboratories), ICAM-1 (15.2, FITC-labeled, Calbiochem), DC-SIGN (eB-h209, Rat IgG2a,k, APC-labeled, eBioscience), CD11c (Bly-6, mouse IgG1, PE-labeled, Becton Dickinson), CD40 (5C3, mouse IgG1,k, Cy-chrome-labeled, Becton Dickinson), CD123 (mouse IgG1, k, PE-labeled, Becton Dickinson), CD83 (HB15e, mouse IgG1, k, R-PE-labeled, Becton Dickinson). The corresponding isotype control antibody was also included in each staining condition. After two washes, the cells were resuspended and fixed in 1% paraformaldehyde in PBS and analyzed using a FACSCalibur flow cytometer and the CELLQUEST program (Becton Dickinson). Live cells were gated by the forward and side light scatter characteristics, and the percentage of positive cells and the mean fluorescence intensity (MFI) of the population were recorded.

RNA isolation, labeling and array hybridization. After infection with retroviral or adenovirus vectors, the cells were harvested and lysed with Trizole (Invitrogen/Life Technologies, Carlsbad, Calif.). Total RNA was isolated, labeled and prepared for hybridization to the Atlas Array filters according to the manufacturer's protocol (Clontech). Hybridization was carried out overnight with 15 ug of labeled cDNA product. After hybridization and washing, the array filters were scanned using a phosphorimager (Storm 486, Molecular Dynamics) and quantitatively analyzed using the Clontech Atlas Array image analysis software.

Semi-quantitative and quantitative RT-PCR analysis of IL-4, IL-10 and IL-12. DC were transduced with LVs and matured as described above. The total RNA was purified using Tri-reagent. For semi-quantitative RT-PCR, Standard one-step RT-PCR (Promega) was performed using primers for human IL-4, IL-10 and IL-12 and the control primers for human GAPDH. For quantitative RT-PCR analysis, the total RNA of DC was isolated by using the Trireagent kit and transcribed into first strand cDNA using oligo-dT and AMV reverse transcriptase, and Real-time RT-PCR was performed on an ABI-Prism 7000 PCR cycler (Applied Biosystems, Foster City, Calif.). The validated PCR primers for IL-12p40, IL-10, GAPDH and the TaqMan MGB probes (6FAM-labeled) were purchased from ABI. PCR mix was prepared according to the manufacturer's instructions (Stratagene and ABI) and thermal cycler conditions were as follows: 1×95° C. 10 min, 40-50 cycles denaturation (95° C. 15 s) and combined annealing/extension (60° C. 1 min). Relative quantification was performed by comparison of threshold cycle values of samples with serially diluted standards.

Preparation of naive CD4+ T cells. CD4⁺ T cells were prepared from PBMC by negative selection using a CD4⁺ T cell isolation Rosette cocktail (StemCell Technologies) according to the manufacturer's instruction. Briefly, In a sterile 200 ml Falcon centrifuge tube, 45 ml buffy coat (approximately 5×10⁸ PBMC) were incubated with 2.25 ml CD4⁺ T cell enrichment Rosette cocktails at 25° C. for 25 min. Thereafter, 45 mL of PBS containing 2% FBS was added to dilute the buffy coat. After gentle mixing, 30 ml of the diluted buffy coat was transferred and layered on top of 15 mL Ficoll Hypaque in a 50 ml Falcon tube, and centrifuged for 25 min at 1,200 g. Non-rosetting cells were harvested at the Ficoll interface and washed twice with PBS (2% FBS), counted, and cryopreserved in aliquots in liquid N₂ for future use. The purity of the isolated CD4⁺ T cells was consistently above 95%. CD4⁺CD45RA naïve T cells were purified based on negative selection of CD45RO⁻ cells using the MACS (Miltenyi Biotec) magnetic affinity column according to the manufacturer's instruction.

In vitro induction of Th functions and intracellular cytokine staining. The in vitro DC:T cell coculture method was according to Caron G, et al. (J. Immunol, 167:3682-3686, 2001). Briefly, purified naïve CD4 T cells were co-cultured with allogeneic mature DC at different ratios (20:1 to 10:1) in serum-free AIM-V media. On day 5, 50 u/ml of rhIL-2 was added, and the cultures were expanded and fed with rhIL-2 containing AIM-V medium every other day for up to 3 weeks. After day 12, the quiescent Th cells were washed and re-stimulated with PMA (10 ng/ml or 0.0162 uM) and ionomycin (1 ug/ml, Sigma-Aldrich) for 5 h. Brefeldin A (1.5 ug/ml) was added during the last 2.5 h of culture. The cells were then fixed, permeablized, stained with FITC-labeled anti-IFN-γ and PE-labeled anti-IL-4 mAb (PharMingen), and analyzed in a FACSCalibur flow cytometer (BD Biosciences).

DC-mediated mixed lymphocyte reaction (MLR). Serial dilutions of DC, from 10,000 cells per well to 313 cells per well, were cultured with 1×10⁵ allogeneic CD4 T cells in 96-well U-bottomed plate in total 200 ul for 5 days. The proliferation of T cells was monitored by adding 20 ul of the CellTiter96 solution to each well according to the manufacturer's instruction (Promega), and the OD reading at 490 nm was obtained.

LVs construction and production. Plasmid construction. The oncoretroviral (MLV) and LVs (HIV-1 and HIV-1 SIN) used for this study were constructed as described previously (Zais et al., J. Virol. 76:7209-7219, 2002). All HIV-1 SIN vectors (pTY) have a 3′ bovine growth hormone polyadenylation signal (bGHpA) inserted behind the 3′ truncated long terminal repeat (LTR). An enhanced green fluorescent protein (eGFP) expression plasmid, pHEFeGFP, was constructed by ligating the NotI-digested pHEF with a NotI-digested eGFP fragment derived from the humanized eGFP construct obtained from the Vector Core of UF Powell Gene Therapy Center. The pTYEFeGFP was made by inserting an eGFP fragment (XhoI-EcoRI) from pTVdl.EFeGFP into pTYEFnlacZ, replacing the nuclear lacZ (nlacZ) gene. pTVdl.EFeGFP was generated by replacing the nlacZ fragment (XhoI-EcoRI) of pTVdl.EFnlacZ with the eGFP fragment (XhoI-EcoRI) isolated from pHEFeGFP. The MLV gag-pol construct was based on pcDNA3.1/Zeo(+) (Invitrogen) with the cytomegalovirus immediate-early promoter replaced by the human elongation factor 1α (EF1α) promoter. The lentiviral vectors expressing cytokine genes or T cell costimulatory genes were constructed by inserting the cDNA encoding these genes into pTYF-EF transducing vector behind the EF1a promoter as described above.

### Example 2

### Results

cDNA microarray analysis of cellular responses following viral transduction. Cellular responses to viral transduction were analyzed by comparing different viral vectors including HIV-1 (LVs), Moloney murine leukemia virus (MLV) and adenoviral (Ad) vectors, in primary human umbilical vein endothelial cells (HUVEC). Both HIV-1 and MLV vectors were prepared by DNA co-transfection and no viral genes were included in the vector genomes as previously described (Chang and Gay, Current Gene Therapy, 1:237-251, 2001; Zaiss et al, supra). The Ad vectors were based on an E1A-deleted vector system which contains most of the adenoviral genes (Graham and Prevec, Manipulation of adenovirus vectors, Vol. 7, Chapter 11, pp. 109-128, 1991). HUVEC were maintained at low passage (<5) and transduced at a multiplicity of infection (moi) of 2-3. To minimize the variables arising from the packaging cells and the transgenes, all three viral vectors used in this study carried a lacZ reporter gene and were produced in 293 cells. The cellular responses of HUVEC were studied using a set of four Clontech Human Atlas Array 1.2 blots each containing 1,176 human cDNAs, nine housekeeping control cDNAs and negative controls.

HUVEC were transduced with mock (control 293 supernatants), LVs, MLV and Ad vectors. The total polyA⁺ RNA was harvested 24 h after infection, labeled with ³²P-dATP by reverse transcription, and used to hybridize to four identical. Clontech Atlas Human Array 1.2 cDNA blots. The results were analyzed using the Clontech AtlasImage 1.5 software and pairwise-comparison. The up- or down-regulated genes were arbitrarily determined by any registered changes of more than 2 fold or above 10,000 signal intensity using the software, and confirmed by visual comparison. The results were summarized into six groups of gene pools arbitrarily set by Clontech: cell cycle and oncogenes, signal transduction, apoptosis and GTPase, transcription and surface signaling, adhesion-receptors-chemokines, and stress responses-interleukins-interferons. See Table 1 below. LVs appeared to enhance transcriptional and surface signaling genes more often than MLV and Ad vectors, and interestingly, IL-10, an immunosuppressive cytokine, was up-regulated after MLV and LVs transduction.

TABLE 1
Effects of viral transduction on gene expression in HUVEC.

| | Ad | MLV | LVs |
|---|---|---|---|
| A: Cell cycle/Oncogenes | ↑ | ↑↑ | ↑↑ |
| B: Signal transduction | ↓ | ↓ | ↓ |
| C: Apoptosis, GTPase | — | — | — |
| D: Transcription, surface signaling | ↑↑ | ↑ | ↑↑↑ |
| E: Adhesion, receptors, chemokines | ↑↑ | ↑↑ | ↑↑ |
| F: Stress response, ILs, IFNs | ↑ | ↑ | ↑ |

The six arbitrarily defined functional genes are shown with fold of changes in gene expression illustrated by up-regulation (↑), down-regulation (↓) or unchanged (—).

Analyses of DC surface marker expression after LVs transduction. Surface marker expression on DC after LVs transduction using different antibodies and flow cytometry. The peripheral blood monocyte (PBM)-derived immature DC were transduced with vectors including mock (control 293 supernatants), empty LVs particles (particles containing HIV-1 capsids and VSV-G envelops without viral genome), LVs, and MLV. The empty LVs was also tested in order to see if viral proteins present in the vector particles could induce changes in DC phenotypes. After treated with LPS plus TNF-α for 24 h, the DC were harvested for antibody staining and flow cytometry. The results are summarized in Table 2. Among the surface molecules tested, CD1a, CD80, CD86, ICAM-1 and DC-SIGN were down-regulated after LVs transduction, but not when empty LVs or MLV was used. The same result was obtained when different preparations of LVs carrying PLAP or Cre reporter genes were tested.

TABLE 2
Surface marker profile of DC transduced with LVs or MLV.

| Surface | Geometrical Mean Fluorescence ± SD | | | |
|---|---|---|---|---|
| Marker | Mock | Empty LVs | LVs-PLAP | MLV |
| CD11c | 48.8 ± 3.2 | 47.2 ± 1.3 | 52.3 ± 2.3 | 55.3 ± 1.1 |
| CD123 | 13.0 ± 0.4 | 13.4 ± 0.8 | 14.9 ± 0.6 | 15.7 ± 0.1 |
| CD1a | 27.3 ± 1.1 | 27.6 ± 2.9 | 21.5 ± 0.2* | 31.0 ± 0.3 |
| CD40 | 8.6 ± 0.1 | 8.9 ± 0.6 | 8.6 ± 0.1 | 9.0 ± 0.3 |
| ICAM-1 | 462.6 ± 57.5 | 376.5 ± 30.1 | 179.5 ± 3.4*** | 498.5 ± 6.9 |
| CD62L | 3.3 ± 0.1 | 3.2 ± 0.03 | 3.7 ± 0.1 | 3.3 ± 0.4 |
| CD80 | 9.9 ± 0.9 | 10.6 ± 0.7 | 9.3 ± 0.2* | 11.3 ± 0.4 |
| (B7-1) | | | | |
| CD83 | 5.8 ± 0.3 | 5.8 ± 0.1 | 6.4 ± 0.01 | 6.0 ± 0.3 |
| CD86 | 39.6 ± 3.5 | 39.6 ± 2.5 | 31.4 ± 0.4* | 47.3 ± 1.5 |
| (B7-2) | | | | |
| DC- | 62.7 ± 4.5 | 55.7 ± 0.4 | 50.6 ± 1.5* | 68.6 ± 4.1 |
| SIGN | | | | |
| HLA- | 13.9 ± 1.3 | 15.8 ± 1.0 | 14.6 ± 0.3 | 17.2 ± 0.9 |
| ABC | | | | |
| HLA-DR | 31.5 ± 0.8 | 28.6 ± 2.2 | 26.9 ± 0.4 | 33.2 ± 1.7 |

Results are presented as geometrical mean fluorescence after flow cytometry.
Asterisks (*) denote significance of difference by Student t-test (*P < 0.05, **P < 0.01, ***P < 0.001).

LVs transduction imparied DC-mediated Th1 immunity. An in vitro DC functional assay using human DC and naïve T cells was performed. DC were generated from PBM in culture with GM-CSF and IL-4, and the PBM-derived day 5 (d5) DC were infected with LVs carrying a PLAP reporter gene. The infected DC were analyzed for PLAP activity on day 7. Under this condition, more than 90% DC were transduced with LVs at moi ˜30-80. To see if IL-10 expression was affected in DC after LVs infection, day 5 DC were infected with LVs and treated the DC with LPS on day 6, and analyzed for IL-10 expression by intracellular cytokine staining (ICCS) using anti-IL-10 monoclonal antibody and flow cytometry on the following day. Similar to LVs transduction of HUVEC, up-regulation of IL-10 in DC was observed after LVs infection.

To further characterize the function of DC after LVs infection, naïve CD4⁺ T cells were purified from peripheral blood mononuclear cells (PBMC) and co-cultured with allogeneic PBM-derived DC after TNF-α and LPS induced maturation. These DC were infected with LVs or MLV on day 5, induced to mature, and co-cultured with the naive CD4⁺ T cells. These T cells were allowed to expand and rest after DC priming for more than 7 days. To analyze Th response, the resting T cells were reactivated on day 7 and day 9 after coculture with ionomycin and PMA and subjected to intracellular staining (ICCS) using antibodies against IFN-γ and IL-4 as described above. The results demonstrated that the IFN-γ-producing Th1 cell population was dramatically reduced, from 72% on day 7, and 75% on day 9 for the control to 27% on day 7 and 22% on day 9 for the LVs-transduced DC, while the Th2 population remained unchanged. A similar but less striking effect was observed for MLV-transduced DC.

Modifications of DC immunity by LVs encoding immune modulatory genes. The cDNA of human CD80 and CD86 was cloned into LVs as depicted in FIG. 1. DC were transduced with LVs carrying a reporter gene (LVs-PLAP), the CD80 cDNA (LVs-CD80) or the CD86 cDNA (LVs-CD86), and treated with LPS and TNF-α 12 hr later. The transduced DC were analyzed for CD80 and CD86 expression by flow cytometry using anti-CD80 and anti-CD86 antibodies 36 h after LVs transduction. Both CD80 and CD86 expression was reduced after LVs-PLAP infection, from 41% to 35% for CD80, and from 61% to 49% for CD86. The expression of CD80 and CD86, however, was up-regulated after transduction with LVs encoding CD80 (from 35% to 44%) and CD86 (from 49% to 76%), respectively.

In other experiements, DC transduced with mock, LVs-PLAP, LVs-PLAP plus LVs-CD80 or LVs-PLAP plus LVs-CD86 were co-cultured with naïve CD4 T cells. After 8 days, the T cells were reactivated and analyzed using anti-IL-4 and anti-IFN-γ antibodies by ICCS and flow cytometry as described above. The results showed that after LVs transduction, the Th1 population was reduced from 24% to 13%, and this impairment could not be corrected by up-regulation of CD80 and CD86 in DC (from 13% to 12% and 13%, respectively).

In other experiements, whether Th1 activation function of DC could be enhanced by supplementing soluble IL-12 and/or FL to the DC culture was investigated where these cytokines were added individually or together to the DC culture throughout viral transduction and the DC:T cell co-culture. The co-cultured T cells were re-activated on day 6 and day 7 for Th analysis. Results of both day 6 and day 7 analyses of the T cells by IL-4 and INF-γ ICCS confirmed the impaired Th1 response after LVs infection (from 37.5% and 20% to 15.6% and 10%, respectively). However, supplementing exogenous IL-12 only partially corrected the impaired Th1 response (from 15.6% and 10% to 19.1% and 11.7% respectively, for IL-12 alone and to 18.7% and 13.2% for IL-12+FL), and FL alone had no effect (from 15.6% and 10.0% to 14.6% and 8.8%, respectively). In other experiments using higher concentrations of soluble IL-12, the impaired Th1 response was fully corrected.

To engineer DC with enhanced endogenous expression of critical cytokines, LVs encoding different cytokines including FL, IL-7, CD40L, bi-cistronic IL-12, and tri-cistronic IL-12/GM-CSF were constructed and tested (FIG. 1). DC were transduced with LVs carrying a reporter gene alone, or co-transduced with LVs expressing different cytokines. The Th functions of the LVs-transduced DC were studied by DC:T cell coculture assay, and 12 days later, the T cells were reactivated as described above, and analyzed by ICCS and flow cytometry. The results showed that LVs reporter vector transduction alone led to reduced Th1 development (from 54.6% to 37.7%/). However, co-transduction of DC with LVs encoding bicistronic IL-12, tricistronic IL-12/GM-CSF, and IL-7, effectively enhanced Th1 response, from 37.7% to 56.2%, 56.2% and 50.7%, respectively. LVs encoding other immune regulatory genes such as FL, GM-CSF, or CD40L did not exhibit any correction effect.

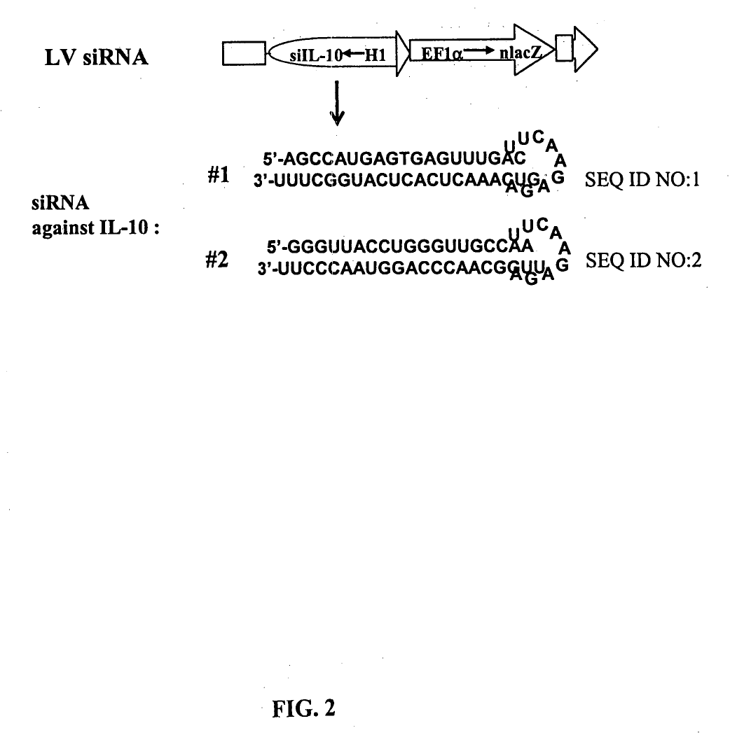

Modulation of DC function by LVs expressing small interfering RNA targeting IL-10. LVs encoding small interfering RNA targeting IL-10 were constructed. Two regions in the IL-10 mRNA were chosen for RNA interference target sites (FIG. 2). The siRNA expression cassette was driven by human H1 pol III promoter and cloned into LVs in the reverse orientation. The LVs-siRNA vector also carried a nlacZ reporter gene adjacent to the pol III siRNA to allow for titer determination. DC were co-transduced with a reporter LVs and the LVs-siRNA targeting IL-10, and then analyzed for IL-10 expression as described above after LPS treatment and ICCS. The results again showed that LVs transduction alone up-regulated IL-10 expression, whereas co-transduction with LVs-siRNA targeting IL-10 down-regulated IL-10 expression. The two IL-10 LVs-siRNA constructs were then compared with LVs-IL-7 in a LVs-co-transduction and DC:T co-culture Th1 functional assay. The co-cultured naïve T cells were activated and rested for 20 days before reactivation and Th cytokine analysis. The results of IL-4 and IFN-γ ICCS demonstrated that both IL-10 LVs-siRNA vectors enhanced Th1 response, and the #2 IL-10 LVs-siRNA displayed enhanced Th1 response at levels comparable to or higher than that of LVs-IL-7. This was further verified with analysis of another Th1 cytokine TNFα ICCS.

### OTHER EMBODIMENTS

It is to be understood that while the invention has been described in conjunction with the detailed description thereof, the foregoing description is intended to illustrate and not limit the scope of the invention, which is defined by the scope of the appended claims. For example, agents that overcome LV-induced DC impairment might be introduced into a target DC using non-lentiviral methods, e.g., using other viral vectors or non-vector based methods. Other aspects, advantages, and modifications are within the scope of the following claims.

What is claimed is:

1. A nucleic acid comprising a first nucleotide sequence derived from a lentivirus and a second nucleotide sequence of non-lentiviral origin that encodes an agent capable of modulating a dendritic cell's ability to activate a T cell.

2. The nucleic acid of claim 1, wherein the second nucleotide sequence encodes IL-7.

3. The nucleic acid of claim 1, wherein the second nucleotide sequence encodes IL-12.

4. A nucleic acid comprising a first nucleotide sequence derived from a lentivirus and a second nucleotide sequence of non-lentiviral origin that encodes an siRNA.

5. The nucleic acid of claim 4, wherein the siRNA is specific for IL-10.

6. The nucleic acid of claim 4, wherein the nucleic acid is comprised within a lentiviral vector.

7. The nucleic acid of claim 6, wherein the lentiviral vector is comprised within a virion.

8. A dendritic cell into which has been introduced a purified nucleic acid comprising a nucleotide sequence that encodes an agent capable of modulating the dendritic cell's ability to activate a T cell.

9. The dendritic cell of claim 8, wherein the cell comprises a lentiviral vector.

10. The dendritic cell of claim 8, wherein the nucleotide sequence encodes IL-7.

11. The dendritic cell of claim 8, wherein the nucleotide sequence encodes IL-12.

12. A dendritic cell into which has been introduced a purified nucleic acid comprising a nucleotide sequence that encodes an agent capable of modulating the dendritic cell's ability to activate a T cell,
wherein the nucleotide sequence encodes an siRNA.

13. The dendritic cell of claim 12, wherein the siRNA is specific for IL-10.

14. The dendritic cell of claim 12, wherein the cell comprises a lentiviral vector.

15. A method of modulating the T cell activating activity of a dendritic cell, the method comprising the step of modulating the amount of at least one cytokine associated with the dendritic cell.

16. The method of claim 15, wherein the at least one cytokine is selected from the group consisting of IL-7, IL-10, and IL-12.

17. The method of claim 16, wherein the amount of IL-7 associated with the cell is increased.

18. The method of claim 16, wherein the amount of IL-12 associated with the cell is increased.

19. A method of modulating the T cell activating activity of a dendritic cell, the method comprising the step of decreasing the amount of IL-10 associated with the dendritic cell.

20. The method of claim 15, wherein the step of modulating the amount of at least one cytokine associated with the dendritic cell comprises contacting the cell with a soluble cytokine.

21. A method of modulating the T cell activating activity of a dendritic cell, the method comprising the step of modulating the amount of at least one cytokine associated with the dendritic cell,
wherein the step of modulating the amount of at least one cytokine associated with the dendritic cell comprises introducing into the dendritic cells a purified nucleic acid comprising a nucleotide sequence that encodes an agent selected from the group consisting of IL-7, IL-12, and an siRNA specific for IL-10.

22. A nucleic acid comprising a first nucleotide sequence derived from a lentivirus and a second nucleotide sequence that encodes an siRNA.

23. A method for modulating expression of a gene in a dendritic cell, the method comprising the step of introducing into the dendritic cell a nucleic acid comprising a first nucleotide sequence derived from a lentivirus and a second nucleotide sequence that encodes an siRNA.


SEQUENCE LISTING (12 sequences)

SEQ ID NO: 1 - RNA, 53 nt, HOMO SAPIENS
agccaugagugaguuugacuucaagagagucaaacucacucacucauggcuuu

SEQ ID NO: 2 - RNA, 49 nt, HOMO SAPIENS
ggguuaccuggguugccaauucaagagauuggcaacccagguaacccuu

SEQ ID NO: 3 - DNA, 32 bp, ARTIFICIAL
  OLIGONUCLEOTIDE
tttctagaccaccatgacagtgctggcgccag

SEQ ID NO: 4 - DNA, 26 bp, ARTIFICIAL
  OLIGONUCLEOTIDE
aaggatcctcagtgctccacaagcag

SEQ ID NO: 5 - DNA, 31 bp, ARTIFICIAL
  OLIGONUCLEOTIDE
tttctagaccaccatgatcgaaacatacaac

SEQ ID NO: 6 - DNA, 32 bp, ARTIFICIAL
  OLIGONUCLEOTIDE
ttgaattcttatgttcagagtttgagtaagcc

SEQ ID NO: 7 - DNA, 30 bp, ARTIFICIAL
  OLIGONUCLEOTIDE
aagcggccgccaccatgttccatgtttctt

SEQ ID NO: 8 - DNA, 33 bp, ARTIFICIAL
  OLIGONUCLEOTIDE
ttctcgagttatcagtgttctttagtgcccatc

SEQ ID NO: 9 - DNA, 64 bp, ARTIFICIAL
  OLIGONUCLEOTIDE
gatccccagccatgagtgagtttgacttcaagagagtcaaactcactcatggcttttttggaaa

SEQ ID NO: 10 - DNA, 64 bp, ARTIFICIAL
  OLIGONUCLEOTIDE
agcttttccaaaaaagccatgagtgagtttgactctcttgaagtcaaactcactcatggctggg

SEQ ID NO: 11 - DNA, 64 bp, ARTIFICIAL
  OLIGONUCLEOTIDE
gatccccgggttacctgggttgccaattcaagagattggcaacccaggtaaccctttttggaaa

SEQ ID NO: 12 - DNA, 64 bp, ARTIFICIAL
  OLIGONUCLEOTIDE
agcttttccaaaaagggttacctgggttgccaatctcttgaattggcaacccaggtaacccggg